(19) United States
(12) Patent Application Publication
(10) Pub. No.: US 2025/0108179 A1
(43) Pub. Date: Apr. 3, 2025

(54) LOSS PREVENTION ACCESSORY FOR MEDICAL DEVICES

(71) Applicant: Jo A. Dowell, High Point, NC (US)

(72) Inventor: Jo A. Dowell, High Point, NC (US)

(21) Appl. No.: 18/904,865

(22) Filed: Oct. 2, 2024

Related U.S. Application Data
(60) Provisional application No. 63/587,357, filed on Oct. 2, 2023.

(51) Int. Cl. A61M 15/00; G08B 21/24

(52) U.S. Cl. CPC A61M 15/0001; G08B 21/24; A61M 2205/581; A61M 2209/06

(57) ABSTRACT

An accessory for storing and securing a medical device, such as a spacer for a metered dose inhaler. The accessory includes a container defining a first end, a second end, and an interior compartment. The container is formed with at least one layer of fabric material. The interior compartment is sized and shaped to receive the medical device. One or both of the ends of the container may include a fastener for selectively opening and closing the ends. An extension ring may be integrally formed with the container or may be a separate component secured to a portion of the container. An electronics assembly may be sewn between a layer or between multiple layers. The electronics assembly may include a battery source, a programmable logic controller, a speaker, and a transceiver. The electronics assembly may be configured with hardware to transmit a signal or emit an auditory notification.

This non-provisional patent application claims all benefits under 35 U.S.C. § 119 (e) of pending U.S. provisional patent application Ser. No. 63/587,357 filed Oct. 2, 2023, entitled “Loss Prevention Accessory for Medical Devices” in the United States Patent and Trademark Office, which is incorporated by reference in its entirety herein.

### FIELD OF DISCLOSURE

The present disclosure relates to security accessories for medical devices, and more specifically, relates an accessory configured to secure medical devices used for inhaler therapy while simultaneously allowing for use of the medical devices.

### DESCRIPTION OF THE PRIOR ART AND OBJECTIVES OF THE PRESENT DISCLOSURE

As would be understood, a metered dose inhaler (MDI) holds medication in a pressurized canister. MDIs are medical devices that deliver medication directly to the lungs of a patient to treat respiratory conditions such as asthma, chronic obstructive pulmonary disease (COPD), and other related diseases. The canister typically sits in a handheld container with a mouthpiece. When a user presses on the canister, a propellant propels a predetermined amount of medicine out of the mouthpiece. The user breathes the predetermined amount of medicine in through their mouth to pull the medicine into their lungs.

Asthma is a reactive airway disease that is disruptive and costly chronic disease imposing a significant burden on children, families, and communities. Currently, there are 5.1 million in the U.S. children under the age of 18 year with asthma. Childhood asthma is the most common health-related issue in children. Asthma accounts for more than 13.8 million missed school days. Minority children with a chronic illness such as asthma experience disproportionate morbidity, in part because of limited resources. For underserved children and their families, significant barriers exist to achieving optimal self-management such as medication and device cost, clinic hours, transportation issues, and competing family demands. Asthma management is heavily influenced by a series of resources. One factor that may negatively affect optimal asthma symptom management for children is access to medications and devices that help to distribute the medications.

Many patients, some of them children, fail to use their inhalers properly. Typically, these users struggle to deliver the medicine to the lungs and instead end up in the mouth and throat. This mis delivery leads to side effects such as sore throat and oral infections. To remedy this, healthcare providers prescribe the use of an AeroChamber to be used with the MDI. An Aerochamber is a type of MDI spacer used to improve medication delivery to the lungs for patients with asthma or chronic obstructive pulmonary disease (COPD) and is designed to help patients inhale metered-dose inhalers correctly. As shown in FIGS. 1-3, an MDI and spacer is a tube-like plastic or metal device placed between an MDI and the patient's mouth and may include a mask. The antistatic valved holding chamber is designed to help improve medication delivery from an MDI using a spacer and a mask system. This improvement is achieved by trapping the medication released from the MDI in a chamber, which the patient inhales through a mouthpiece. This allows more of the medication to reach the lungs, improving its effectiveness and reducing potential side effects.

The Aerochamber is just one example of a spacer device combined with MDI inhalers to improve medication delivery for patients with respiratory diseases. Other examples include valved holding chambers (VHCs) and breath-actuated inhalers. VHCs are larger and include a one-way valve that helps prevent medication loss during exhalation. Breath-actuated inhalers are designed to deliver medication automatically when the patient takes a deep breath, eliminating the need for coordination between inhalation and medication release.

The global MDI market is expected to grow in the coming years, driven by factors such as the rising demand for effective and convenient treatment options, advancements in inhalation technology, and increased awareness and diagnosis of respiratory diseases. AeroChambers and other MDI-related spacers are expensive. In particular, the more advanced spacers with additional features such as one-way valves or digital monitoring capabilities are even more costly. Accordingly, the cost to replace lost and missing medical devices is extremely high. Such costs are especially burdensome on low-income and delays in receiving therapy create high risk situations. Due to the prevalence of respiratory diseases diagnosed in children, respiratory therapy must accompany children with such diagnoses. Parents and children are likely to misplace or lose such medical devices. Further, in cases where loss of the medical device has occurred without the patient's awareness, the patient may be placed in a high-risk situation where they are in severe need of the medical device while it is missing. Accordingly, there is a need for a loss prevention accessory to securely store medical devices when not in use. There is further need for the accessory to be convenient and readily accessible so medical interventions may be quickly accessed.

Although it may be standard to prescribe the medication with a spacer, the prescription typically only includes one spacer device. If the spacer device is misplaced, the parent would have to pay out of pocket to replace the spacer. Insurance companies often only pay for one spacer and will not replace misplaced spacers. Accordingly, there is a need for a loss prevention accessory to securely store medical devices when not in use. There is further need for the accessory to be convenient and readily accessible so medical interventions may be quickly accessed.

Thus, in view of the problems and disadvantages associated with prior art devices, the present disclosure was conceived and one of its objectives is to provide a sleeve-like container for storing and preventing loss of a medical device, the sleeve-like container formed of at least one fabric layer defining an interior compartment sized and shaped to receive a medical device, the sleeve like container defining a first end and a second end, at least one of the ends being selectively openable and closeable.

It is another objective of the present disclosure to provide a sleeve-like container including an interior layer and an exterior layer of fabric material defining an interior compartment sized and shaped to receive a medical device, a first end, and a second end, and a location tracking device sewn between the interior layer and the exterior layer, wherein either the first end or second end, or both ends are selectively openable and closeable.

It is still another objective of the present disclosure to provide a sleeve-like container including at least one layer of fabric defining an interior compartment sized and shaped to receive a medical device, a first end, and a second end, and a speaker device sewn within the layer of fabric, wherein the speaker device includes and is electrically connected to a battery and a programmable logic controller.

It is yet another objective of the present disclosure to provide a sleeve-like container including of at least one layer of fabric defining an openable end, a permanently closed end, and an interior compartment for receiving a medical device, the container further including an electronics assembly securely to the at least one layer of fabric, the electronics assembly including a battery source, a programmable logic controller, a speaker, and a transceiver all electrically connected.

It is a further objective of the present disclosure to provide a sleeve-like container including an interior layer and an exterior layer of fabric sewn together, the layers defining a first end and a second end, both ends selectively openable, and an interior compartment for receiving a medical device, the container further including an electronic assembly configured with hardware programmed to emit an auditory notification when at least one of the ends is opened.

It is still a further objective of the present disclosure to provide an accessory for a medical device, the accessory defining a sleeve-like container including an exterior layer of fabric material, an interior layer of fabric material, an electronic assembly including a battery, a programmable logic controller, a transceiver, and a speaker, the electronic assembly positioned between the interior layer and the exterior layer of fabric material.

It is yet a further objective of the present disclosure to provide a sleeve-like container that is easy to use and manufacture.

Various other objectives and advantages of the present disclosure will become apparent to those skilled in the art as a more detailed description is set forth below.

### SUMMARY OF DISCLOSURE

The aforesaid and other objectives are realized by providing an accessory for storing and securing a medical device, such as a spacer for a metered dose inhaler (MDI). The accessory is a sleeve-like container formed of at least one layer of fabric defining a first end and a second end. At least one of said ends is selectively openable and includes a means for fastening the end open. The accessory may further include an electronics assembly sewn within the fabric layer, the electronic assembly may include a battery source, a programmable logic controller, a transceiver, and a speaker all electrically connected to one another. The electronics assembly is configured with hardware to provide location tracking functionality by transmitting and receiving a radio signal to communicate the position of the accessory to a remote device, such as a cell phone.

The fabric material is preferably made of a material selected from the group consisting of cotton, viscose fibers, polyester, polyamide, polypropylene, viscose rayon, rayon, yarns, elastomeric yarns, silk, linen, wool, microfiber, bamboo viscose, and combinations thereof. The select fabric material also preferably has certain material properties selected from the group consisting of hypoallergenic, antimicrobial, compressive, air permeability, moisture permeability, and a combination of such material properties.

In some embodiments, the container may define only a single selectively openable end. In other embodiments, the container may define two selectively openable ends to allow usage of the medical device stored within without having to completely remove the medical device from the interior compartment. Ends configured to selectively open and close include a means for fastening the ends open and closed. The means for fastening the ends open and close are selected from the group consisting of hook and loop fasteners, snaps, buttons, zippers, hooks, ties, and a combination of the previously mentioned fastening means.

### BRIEF DESCRIPTION OF THE DRAWINGS

FIG. 1 shows a photographic perspective view of a prior art metered dose inhaler (MDI).

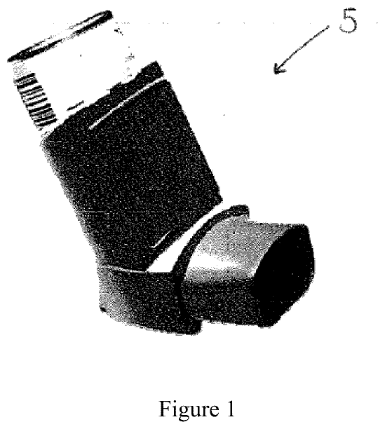

FIG. 2 pictures a photographic perspective view of a prior art spacer and mask for an MDI.

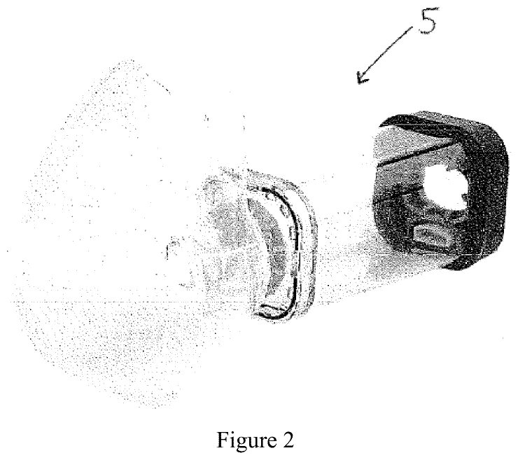

FIG. 3 depicts a photographic elevated side view of the MDI placed in the spacer.

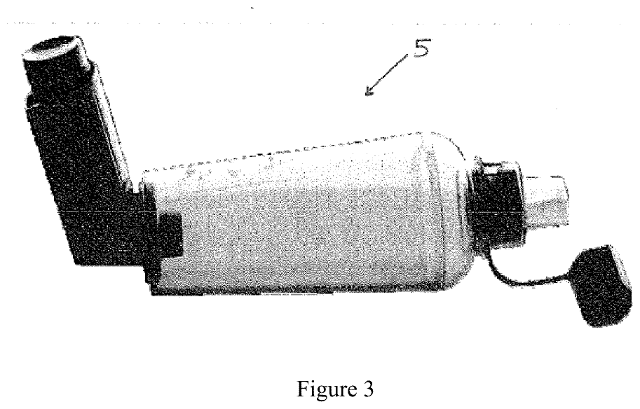

FIG. 4 shows an elevated side exterior view of an embodiment of a loss prevention accessory for a medical device according to the present disclosure.

FIG. 5 is an elevated end view of the spacer and an open end of the loss prevention accessory for a medical device of FIG. 4.

FIG. 6 illustrates a perspective view of the spacer with a portion inserted within the loss prevention accessory for a medical device of FIG. 4.

FIG. 7 demonstrates a perspective view of a portion of the spacer placed within the loss prevention accessory for a medical device of FIG. 4.

FIG. 8 illustrates a photographic elevated end view of the spacer placed within the loss prevention accessory for a medical device of FIG. 4.

FIG. 9 depicts a line drawing of an embodiment of the present disclosure including one selectively openable end, a permanently closed end, and an electronics assembly positioned within a fabric layer.

FIG. 10 demonstrates a line drawing of an embodiment of the present disclosure including a first selectively openable end, a second selectively openable end, and an electronics assembly positioned within a fabric layer.

### DETAILED DESCRIPTION OF THE PREFERRED EMBODIMENT AND OPERATION OF THE PRESENT DISCLOSURE

Various exemplary embodiments of the present disclosure are described below. Use of the term “exemplary” means illustrative or by way of example only, and any reference herein to “the disclosure” is not intended to restrict or limit the disclosure to exact features or step of any one or more of the exemplary embodiments disclosed in the present specification. References to “exemplary embodiment”, “one embodiment”, “an embodiment”, “various embodiments”, and the like may indicate that the embodiment(s) of the disclosure so described may include a particular feature, structure, or characteristic, but not every embodiment necessarily incudes the particular feature, structure, or characteristic. Further, repeated use of the phrase “in one embodiment”, “in an exemplary embodiment”, or “in an alternative embodiment” do not necessarily refer to the same embodiment, although they may.

It is also noted that terms like “preferably”, “commonly”, and “typically” are not utilized herein to limit the scope of the disclosure or to imply that certain features are critical, essential, or even important to the structure or function of the disclosure. Rather, these terms are merely intended to highlight alternative or additional features that may or may not be utilized in a particular embodiment of the present disclosure.

The present disclosure is described more fully hereinafter with reference to the accompanying figures, in which one or more exemplary embodiments of the disclosure are shown. Like numbers used herein refer to like elements throughout. The disclosure may, however, be embodied in many different forms and should not be construed as limited to the embodiments set forth herein; rather, these embodiments are provided so that this disclosure will be operative, enabling, and complete. Accordingly, the particular arrangements disclosed are meant to be illustrative only and not limited as to the scope of the disclosure, and any and all equivalents thereof. Moreover, many embodiments such as adaptations, variations, modifications, and equivalent arrangements will be implicitly disclosed by the embodiments described herein and fall within the scope of the instant disclosure.

Although specific terms are employed herein, they are used in a generic and descriptive sense only and not for the purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. Where only one item is intended, the terms “one and only one”, “single”, or similar language is used. When used herein to join a list of items, the term “or” denotes at least one of the items but does not exclude a plurality of items of the list.

For exemplary methods or processes of the disclosure, the sequence and/or arrangement of steps described herein are illustrative and not restrictive. Accordingly, it should be understood that, although steps of various processes or methods may be shown and described as being in a sequence or temporal arrangement, the steps of any such processes or methods are not limited to being carried out in any particular sequence or arrangement, absent an indication otherwise. Indeed, the steps in such processes or methods generally may be carried out in various different sequences and arrangements while still falling within the scope of the present disclosure.

Additionally, any references to advantages, benefits, unexpected results, or operability of the present disclosure are not intended as an affirmation that the disclosure has previously been reduced to practice or that any testing has been performed. Likewise, unless stated otherwise, use of verbs in the past tense (present perfect or preterit) is not intended to indicate or imply that the disclosure has previously been reduced to practice or that any testing has been performed.

For a better understanding of the disclosure and its operation, turning now to the drawings, the present disclosure is directed towards an accessory 10 for the secure storage and loss prevention of medical devices. In particular, an embodiment of the present disclosure is directed towards an accessory 10 for the storage of MDI, spacers, and other respiratory therapy medical devices 5. In one or more embodiments of the present disclosure, the accessory 10 may store and protect the medical devices 5 when not in use. In other embodiments, the accessory 10 may store and protect the medical devices 5 while allowing for their use by a user.

According to some embodiments, the loss prevention accessory 10 may comprise a sleeve-like container 12. In such embodiments, when the medical device 5 is not positioned within an interior compartment 20 of the sleeve-like container 12, the container 12 may be flattened for improved convenience. In these and other embodiments, one end 14 of the sleeve-like container 12 may be open while the opposite end 16 may be closed. In these and other embodiments, the closed end 16 may be always closed (i.e., sewn closed, and not capable of opening without the end remaining open after being opened without additional materials and/or tools). In other embodiments, the one or both ends 14, 16 may include a fastener 22 for selectively opening and closing the ends 14, 16. Suitable fasteners 22 may include (but are not limited to) hook and loop fasteners, snaps, buttons, zippers, hooks, ties, and a combination of these fasteners.

In some embodiments, the sleeve-like container 12 is made of a fabric layer 18 of material. In one or more alterantive embodiments, the sleeve-like container 12 may comprise one or more layers 18 of fabric, where the layers 18 may be made of the same type of fabric or different types of fabric. In these and other embodiments, the fabric selected defines a relatively high modulus of elongation, is comparatively stretchable or provides a compressive force relative to the surface of the medical device 5, such that it remains snug to the medical device 5 stored within the interior compartment 20. The stretchable fabric advantageously provides for a user to access any medical device 5 stored within the interior compartment 20, while also securing the medical device 5 when not in use. Further advantages include reduced probability of snagging or ripping of the fabric due to a loose fit around the medical device 5.

Fabric suitable for use in the one or more layers 18 of the present disclosure may be woven, knit, or nonwoven. The types of fabric suitable for use in the present disclosure includes without limitation cotton, viscose fibers, polyester, polyamide, polypropylene, polyethylene, viscose rayon, rayon, yarns, elastomeric yarns, silk, linen, wool, microfiber, bamboo viscose, and other known in the art. The fabric used may possess hypoallergenic properties, antimicrobial properties, compressive properties, air permeability, moisture permeability, amongst others. In embodiments of the present disclosure employing one or more layers 18 of fabric, the fabric selected for each layer 18 may be chosen having the same properties or different properties. For example, in some embodiments, the interior layer 18A of fabric, in contact with the medical device 5, may be antimicrobial or hypoallergenic while the exterior layer 18B may be more durable including higher tensile strength, abrasion resistance, or tearing resistance. A multi-lay construction advantageously allows for each layer 18A, 18B to maximize the overall performance of the accessory 10.

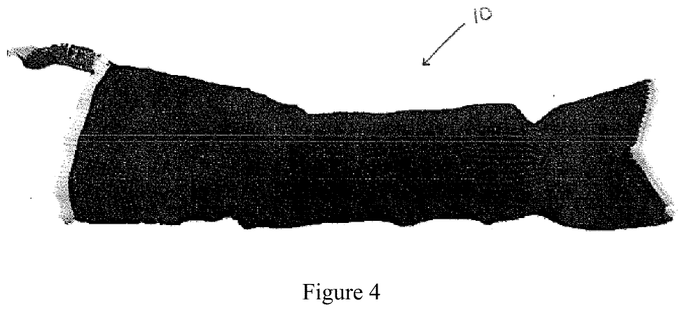

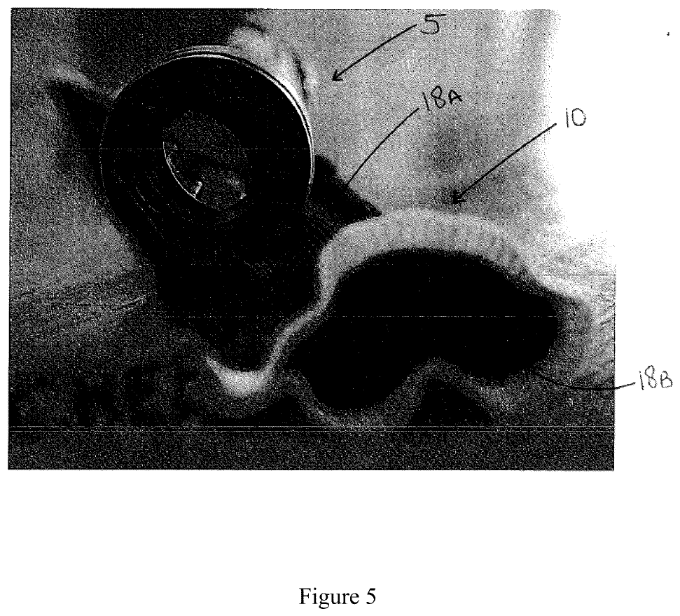

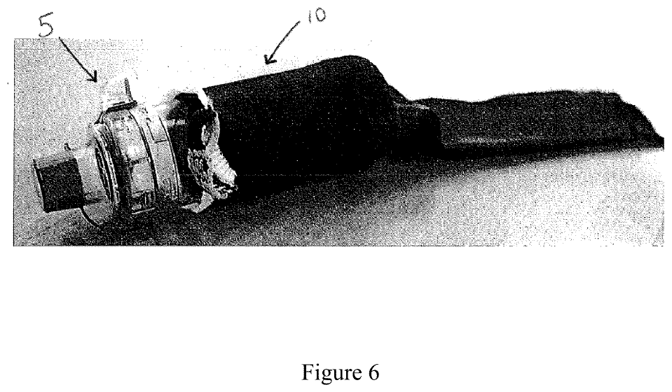

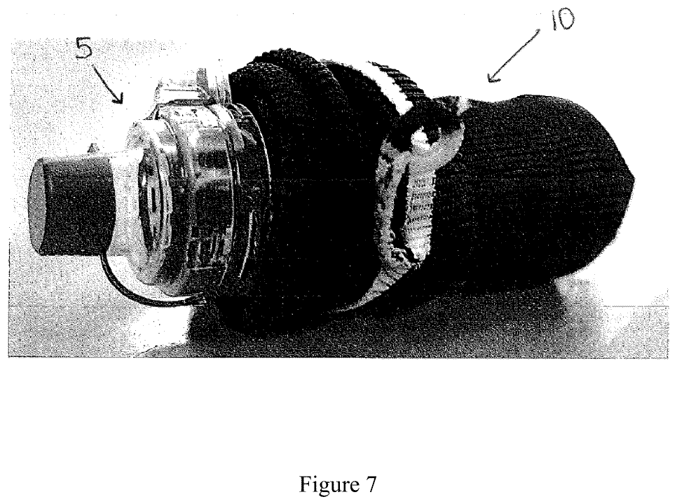

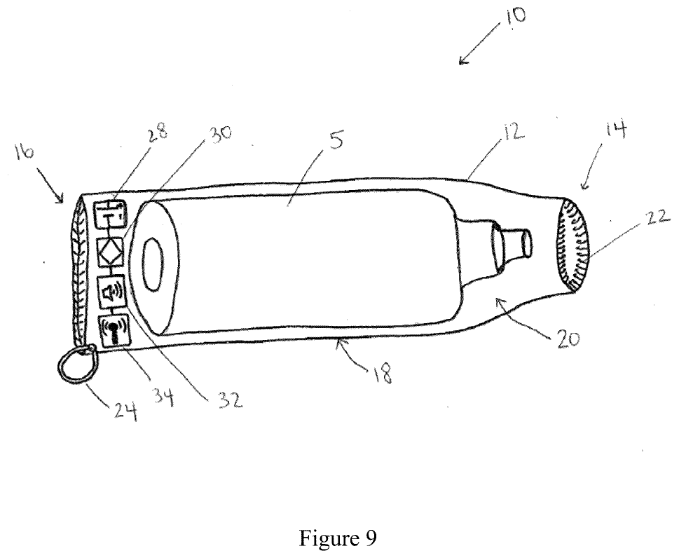

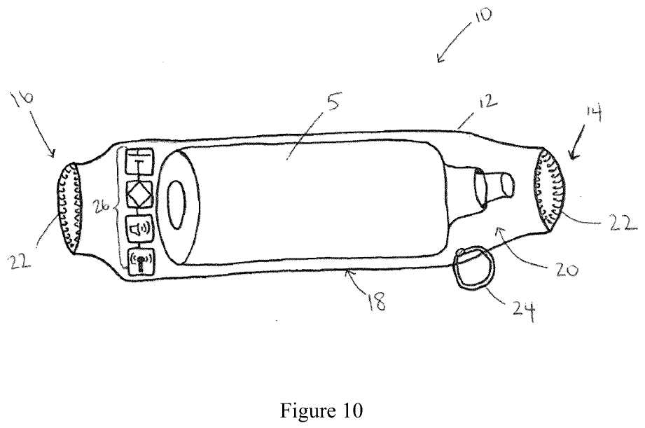

An embodiment of the present disclosure is shown in FIGS. 4-10, demonstrating the accessory storing a medical device 5. In FIG. 4, an embodiment of the loss prevention accessory 10 is shown storing the medical device 5 within the interior compartment 20. As shown throughout the figures, the medical device 5 is a spacer for a MDI. In this embodiment, the sleeve-like container 12 defines the interior compartment 20 that is configured (i.e., sized, shaped, and otherwise capable) to accommodate the physical dimensions of the medical device 5 including its circumference and length. This is shown in FIGS. 5 through 10 where the medical device 5 is placed into the interior compartment 20 of the sleeve-like container 12. As shown in the figures, particularly FIG. 9, one end 14 of the accessory 10 may be stitched closed while the opposite end 16 includes a fastener 22 (hook and loop fasteners shown) for selectively opening and closing the opposite end 16. A ring extension 24 may be secured to the container 12 to advantageously allow the accessory 10 to be attached to a separate bag, purse, backpack, or person responsible for or that requires respiratory therapy. The ring extension 24 may further carry other attachments such as additional storage, key chains with instructions, and/or cleaning accessories. The instructions may include information regarding the owner of the medical device 5, directions for proper use of the accessory 10 and medical device 5. The ring extension 24 may be integrally sewn onto one of the fabric layers 18A, 18B of the sleeve-like container 12, as shown in FIG. 4, FIG. 6, and FIG. 7. The ring extension 24 may also define a metal ring (i.e., key chain ring) that is secured to the sleeve-like container 12 via a hole formed in the one or more fabric layers 18, as shown in FIG. 9 and FIG. 10. As shown in the figures, it is preferable that at least one layer 18 of the container 12 is stretchable so that the fabric is not loose and the accessory snuggly surrounds the medical device 5 stored within the interior compartment 20.

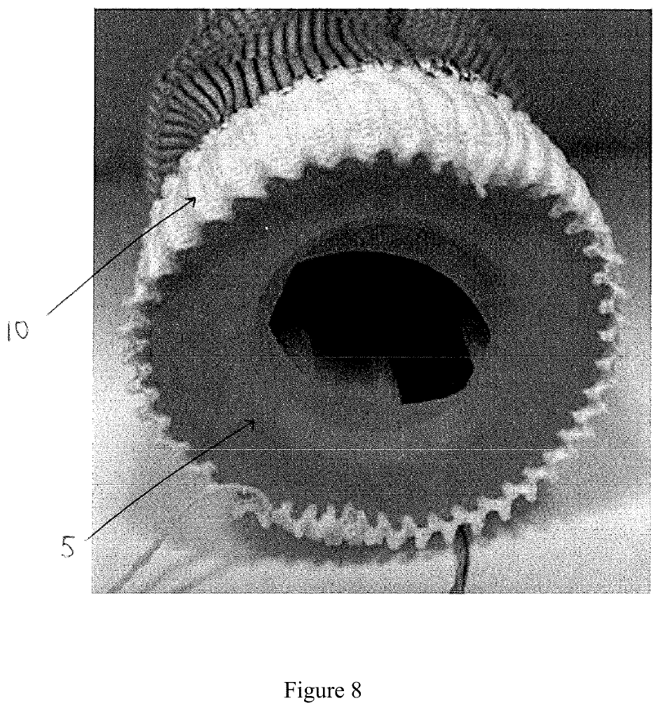

As shown in FIGS. 8 and 10, in embodiments wherein the container 12 defines two ends 14, 16 that include fasteners 22 (hook and loop fasteners shown) to selectively open and close the ends 14, 16, the user may utilize the medical device 5 without completely removing the medical device 5 from the interior compartment 20. In other embodiments, such as the one shown in FIG. 9, the user must remove the medical device 5 from the accessory 10 in order to utilize the medical device 5.

According to some embodiments, the loss prevention accessory 10 may include an electronics assembly 26. The electronics assembly 26 may be sewn into a layer of fabric 18 or may be sewn between the interior layer 18A and exterior layer 18B of fabric. The electronics assembly 26 may include a battery source 28, a programmable logic controller 30 (PLC), a speaker 32, and a transceiver 34. The battery source 28 may be any battery source known in the art capable of providing power to the rest of the components of the electronics assembly 26. The battery source 28 may be single use battery source or may be rechargeable. The programmable logic controller 30 includes a processor for executing instructions stored on a memory. The programmable logic controller 30 is in electronic communication with the battery 28 and the other components of the assembly, such as the speaker 32 and/or the transceiver 34. The speaker 32 is configured to emit an auditory notification in certain situations, such as when the accessory 10 is lost, opened, or during an emergency. The transceiver 34 is configured with hardware to send and receive a signal from another device in certain situations. For example, if the accessory 10 is lost, the transceiver 34 may be configured to receive a signal to be processed by the PLC 30 and cause the speaker 32 to emit an auditory notification to assist the user in locating the accessory 10. In other embodiments, the transceiver 34 may be configured with hardware to transmit a signal to another device, such as a mobile phone, providing the location of the accessory 10.

The illustrations and examples provided herein are for explanatory purposes and are not intended to limit the scope of the appended claims.

I claim:

1. An accessory (10) for retaining and securing a medical device (5), the accessory (10) comprising:
a container (12) including at least one layer (18) of material defining a first end (14), a second end (16), and an interior compartment (20) dimensioned to receive a medical device (5) therein; and
an electronics assembly (26) sewn into the at least one layer (18) of material, the electronics assembly (26) including a battery source (28) electrically connected to a programmable logic controller (30) and transceiver (34),
wherein the electronics assembly (26) is configured with hardware to transmit a location of the accessory (10) via the transceiver (34).

2. The accessory (10) of claim 1, wherein the electronics assembly (26) further includes a speaker (32) device in electrical communication with the other components of the electronic assembly (26), the speaker (32) configured to emit an auditory notification.

3. The accessory (10) of claim 1, wherein the at least one layer (18) of material defines a flexible and stretchable fabric material.

4. The accessory (10) of claim 1, wherein the container (12) includes two layers of material, an interior layer (18A) and an exterior layer (18B).

5. The accessory (10) of claim 4, wherein the interior layer (18A) is made of a material having properties selected from the group consisting of hypoallergenic, antimicrobial, compressive, air permeability, moisture permeability, and a combination of these properties.

6. The accessory (10) of claim 4, wherein the exterior layer (18B) is made of a material having properties selected from the group consisting of hypoallergenic, antimicrobial, compressive, air permeability, moisture permeability, and a combination of these properties.

7. The accessory (10) of claim 1, wherein the material of the least one layer (18) is selected from the group consisting of cotton, viscose fibers, polyester, polyamide, polypropylene, viscose rayon, rayon, yarns, elastomeric yarns, silk, linen, wool, microfiber, bamboo viscose, and combinations thereof.

8. The accessory (10) of claim 1, wherein the first end (14) is permanently closed.

9. The accessory (10) of claim 8, wherein the second end (16) includes a fastener (22) for selectively opening and closing the second end (16).

10. The accessory (10) of claim 10, wherein the fastener (22) is selected from the group consisting of hook and loop fasteners, snaps, buttons, zippers, hooks, ties, and combinations thereof.

11. The accessory (10) of claim 1, wherein the first end (14) and the second end (16) include a fastener (22) for selectively opening and closing the first end (14) and second end (16).

12. The accessory (10) of claim 11, wherein the fastener is selected from the group consisting of hook and loop fasteners, snaps, buttons, zippers, hooks, ties, and combination thereof.

13. The accessory (10) of claim 1 wherein the container further includes a ring extension (24).

14. The accessory (10) of claim 13, wherein the ring extension is integrally sewn onto the at least one layer (18) of material.

15. The accessory (10) of claim 13, wherein the ring extension defines a metal ring.

16. An accessory (10) for retaining and securing a medical device (5), the accessory (10) comprising:
a container (12) including at least one layer (18) of material defining a first end (14), a second end (16), and an interior compartment (20) dimensioned to receive a medical device (5) therein; and
an electronics assembly (26) sewn into the at least one layer (18) of material, the electronics assembly (26) including a battery source (28) electrically connected to a programmable logic controller (30) and speaker (32),
wherein the electronics assembly (26) is configured with hardware configured to emit an auditory notification from the speaker (32).

17. The accessory (10) of claim 16, wherein the electronics assembly (26) further includes a transceiver (34) device in electrical communication with the other components of the electronic assembly (26), electronics assembly (26) configured with hardware to transmit a location of the accessory (10) via the transceiver (34).

18. The accessory (10) of claim 16, wherein the first end (14) is permanently closed.

19. The accessory (10) of claim 18, wherein the second end (16) includes a fastener (22) for selectively opening and closing the second end (16).

20. The accessory (10) of claim 18, wherein the fastener (22) is selected from the group consisting of hook and loop fasteners, snaps, buttons, zippers, hooks, ties, and combinations thereof.
